(19) United States
(12) Patent Application Publication
(10) Pub. No.: US 2023/0333125 A1
(43) Pub. Date: Oct. 19, 2023

(54) METHOD FOR DIAGNOSING NONALCOHOLIC STEATOHEPATITIS

(71) Applicants: NATIONAL UNIVERSITY CORPORATION HOKKAIDO UNIVERSITY, Sapporo-shi, Hokkaido (JP); EIKEN KAGAKU KABUSHIKI KAISHA, Taito-ku, Tokyo (JP)

(72) Inventors: Toshihiro SAKURAI, Hokkaido (JP); Shu-Ping HUI, Hokkaido (JP); Hitoshi CHIBA, Hokkaido (JP)

(73) Assignees: NATIONAL UNIVERSITY CORPORATION HOKKAIDO UNIVERSITY, Sapporo-shi, Hokkaido (JP); EIKEN KAGAKU KABUSHIKI KAISHA, Taito-ku, Tokyo (JP)

(21) Appl. No.: 18/024,955

(22) PCT Filed: Jun. 15, 2021
(86) PCT No.: PCT/JP2021/022648

§ 371 Date: Mar. 6, 2023

(30) Foreign Application Priority Data
Sep. 7, 2020 (JP) 2020-149798

(51) Int. Cl. G01N 33/92

(52) U.S. Cl. CPC G01N 33/92; G01N 2800/085; G01N 2333/775

(57) ABSTRACT

A problem of the present invention is to provide a diagnostic biomarker of nonalcoholic steatohepatitis (NASH) useful for differentiation of simple steatosis (SS) from NASH and use thereof. Specifically, the present invention relates to a diagnostic biomarker of nonalcoholic steatohepatitis used to differentiate nonalcoholic steatohepatitis from simple steatosis comprising the oxidized apolipoprotein A-I (ApoA-I) modified by dioxidation of the 72nd tryptophan in the ApoA-I consisting of the amino acid sequence as shown in SEQ ID NO: 1.

### TECHNICAL FIELD

The present invention relates to a diagnostic biomarker of nonalcoholic steatohepatitis that is useful for differentiation of, for example, simple steatosis from nonalcoholic steatohepatitis and use thereof.

### BACKGROUND ART

In recent years, nonalcoholic steatohepatitis (NASH) has drawn attention as the pathological conditions of fatty liver disease without a history of alcohol consumption. The mechanism of NASH development is as follows. Simple steatosis (SS) is first developed upon accumulation of fats in the liver because of, for example, insulin resistance, and hepatic inflammation or fibrosis takes place upon oxidative stress or the like on SS, which is then led to NASH. Since NASH irreversibly advances to hepatic fibrosis, cirrhosis, and hepatic cancer, prognosis thereof is poor, and it necessitates liver transplantation. A set of pathological conditions of SS and NASH is referred to as “nonalcoholic fatty liver disease (NAFLD).” It is deduced that there are 10,000,000 or more NAFLD patients in Japan, and SS patients account for 80% to 90% of such patients. Prognosis of SS is good. On the other hand, NASH patients account for other 10% to 20%, and it is known that 30% of NASH patients develop cirrhosis or hepatic cancer in 10 years. In Europe and the United States, 30% of adult individuals are reported to have fatty liver, and there are numerous patients and prospective patients in the world. That is, the situation is critical (Non-Patent Literature 1). In daily medical practice, diagnosis of NASH necessitates tissue diagnosis by liver biopsy, which is invasive and painful. Accordingly, development of an optimal diagnostic marker of NASH is in urgent need.

Meanwhile, qualitative study on high-density lipoproteins (HDLs) has drawn attention in recent years. Oxidized HDL (oxHDL) has lost the anti-atherogenic action inherent to HDL, and it induces inflammation or accelerates development of ROS in vascular endothelial cells (Non-Patent Literatures 2 and 3). Also, oxHDL₃ was found to induce the plasminogen activator inhibitor (PAI-1) in endothelial cells and accelerate arterial sclerosis (Non-Patent Literature 4). oxHDL also accelerates inflammation or development of ROS in renal proximal tubule cells and renal mesangial cells (Non-Patent Literatures 5 and 6). The addition of oxHDL to peripheral blood mononuclear cells accelerates foam cell formation and induces inflammation or development of ROS (Non-Patent Literature 7). The concentration of a particular oxidation-modified peptide in the blood in the apolipoprotein A-I (ApoA-I), which is the most abundant protein in HDL, is reported to reflect the pathological conditions of oxidative stress-associated disease, such as coronary disease or diabetes mellitus (Non-Patent Literatures 2 and 8). At the Cleveland Heart Lab. Founded by Dr. Hazen et al., a test method using the monoclonal antibody (r8B5.2) that recognizes ApoA-I containing the oxidized 72nd tryptophan was developed, and clinical trials have been initiated (http://www.clevelandheartlab.com/ and http://tgs.freshpatents.com/Tryptophan-bx1.php). According to another report, the oxHDL/HDL level in the blood becomes significantly high when BMI is over 30 (Non-Patent Literature 9). Among patients with non-diabetic hyperlipidemia, the group with a high fasting blood sugar level exhibited a high oxHDL level, and the correlation between oxHDL and blood sugar and a possibility of oxHDL as a biomarker of coronary disease were suggested (Non-Patent Literature 10). Thus, oxHDL may be useful as a biomarker that reflects oxidative stress-induced disease.

As described above, most reports concerning HDL in functional disorders indicate the correlation thereof with arterial sclerosis. In contrast, the correlation between oxidized HDL and liver disease has not yet been sufficiently studied, and it remains unknown. In the past, the amounts of various proteins were compared between 2 groups by proteomics performed with the use of serum samples obtained from NASH patients and control subjects, and significant differences were observed in 15 types of proteins (Non-Patent Literature 11). Also, significant differences were observed in 2 types of proteins by proteomics performed in the same manner with the use of serum samples obtained from NASH patients and control subjects (Non-Patent Literature 12). According to such analyses, however, protein species useful for differentiation of SS from NASH were not identified.

Patent Literature 1 discloses a method for diagnosis of liver disease (hepatic cell carcinoma or hepatitis B) using, as the indicator, the presence of the ApoA-I isoform having oxidized W50, W108, and M112 residues. However, Patent Literature 1 does not describe that such oxidized ApoA-I isoform is useful for differentiation of SS from NASH.

### PRIOR ART LITERATURES

### Patent Literatures

Patent Literature 1: US 2008/0090223 A

### Non-Patent Literatures

Non-Patent Literature 1: Bellentani S., Liver Int., 37 Suppl. 1: 81-4, 2017

Non-Patent Literature 2: Huang Y. et al., Nature Medicine, 20: 193-203, 2014

Non-Patent Literature 3: Perez L. et al., Lab. Invest., 99: 421-37, 2019

Non-Patent Literature 4: Norata G. D. et al., Brit. J. Haematol., 127: 97-104, 2004

Non-Patent Literature 5: Gao X. et al., Int. J. Mol. Med., 34: 564-72, 2014

Non-Patent Literature 6: Zhang M. et al., Diabetes Metab. Res. Rev., 26: 455-63, 2010

Non-Patent Literature 7: Soumyarani V. S. and Jayakumari N., Mol. Cell Biochem., 366: 277-85, 2012

Non-Patent Literature 8: Brock J. W. C. et al., J. Lipid Res., 49: 847-55, 2008

Non-Patent Literature 9: Peterson S. J. et al., Prostag. Oth. Lipid, M., 123: 68-77, 2016

Non-Patent Literature 10: Kotani K. et al., Clin. Chim. Acta., 414: 125-9, 2012

Non-Patent Literature 11: Bell N. et al., Hepatology, 51: 111-20, 2010

Non-Patent Literature 12: Rodriguez-Suarez E. et al., Proteomics Clin. Appl., 4: 362-71, 2010

### SUMMARY OF THE INVENTION

### Problem to be Solved by the Invention

Under the above circumstances, an object of the present invention is to provide a diagnostic biomarker of nonalcoholic steatohepatitis (NASH) that is useful for differentiation of simple steatosis (SS) from NASH.

### Means for Solving the Problem

To date, there has been no reports concerning the apolipoprotein A-I (ApoA-I) that has been oxidation-modified in a NASH-specific manner, and an extent of oxidation of the protein in the high-density lipoprotein (HDL) in NASH patients has not yet been elucidated.

The present inventors have conducted concentrated studies in order to solve the above problem. As a result, they aimed at identifying oxidation-modified peptides that would increase in a manner specific to HDL derived from NASH patients and performed the analysis (1) and (2): (1) for the purpose of identification of a type of the oxidation-modified peptide in HDL, HDLs derived from healthy subjects were oxidized by metals, and the oxidation-modified peptides were simultaneously analyzed using a mass spectrometer; and (2) the oxidation-modified peptides in HDLs were analyzed and compared among 3 groups; i.e., a group of healthy subjects, a group of SS patients, and a group of NASH patients. As a result, they found that a particular oxidized ApoA-I peptide would serve as a diagnostic biomarker of NASH useful for differentiation of SS from NASH. This has led to the completion of the present invention.

Specifically, the present invention includes the following.
(1) A method of testing for nonalcoholic fatty liver disease comprising a step of measuring the abundance of the oxidized apolipoproteins A-I (ApoA-I) in a biological sample obtained from a patient with nonalcoholic fatty liver disease.
(2) The method according to (1), wherein the step of measurement comprises measuring the abundance of the oxidized ApoA-I modified by dioxidation of the 72nd tryptophan of the ApoA-I consisting of the amino acid sequence as shown in SEQ ID NO: 1 in the oxidized ApoA-I in the biological sample obtained from the patient.
(3) The method according to (1) or (2), which further comprises a step of testing for the nonalcoholic fatty liver disease of the patient to differentiate nonalcoholic steatohepatitis from simple steatosis using, as the indicator, the measured abundance of the oxidized ApoA-I.
(4) The method according to (3), wherein the abundance greater than the abundance in the biological sample obtained from a patient with simple steatosis indicates a higher possibility of having developed nonalcoholic steatohepatitis.
(5) The method according to (3), wherein the indicator is the ratio of the abundance of the oxidized ApoA-I relative to the abundance of total ApoA-I in the biological sample.
(6) The method according to (5), wherein the ratio higher than the ratio in the biological sample obtained from a patient with simple steatosis indicates a higher possibility of having developed nonalcoholic steatohepatitis.
(7) The method according to any one of (1) to (6), wherein the biological sample obtained from the patient is a high-density lipoprotein (HDL) in a blood sample selected from the group consisting of whole blood, serum, and plasma samples.
(8) A diagnostic biomarker of nonalcoholic steatohepatitis used to differentiate nonalcoholic steatohepatitis from simple steatosis comprising the oxidized apolipoprotein A-I (ApoA-I) modified by dioxidation of the 72nd tryptophan in the ApoA-I consisting of the amino acid sequence as shown in SEQ ID NO: 1.

This description includes the contents as disclosed in Japanese Patent Application No. 2020-149798, which is a priority document of the present application.

### Effects of the Invention

According to the present invention, a patient having nonalcoholic fatty liver disease can be diagnosed to have nonalcoholic steatohepatitis with poor prognosis or simple steatosis with good prognosis by a non-invasive test method.

### BRIEF DESCRIPTION OF THE DRAWINGS

FIG. 1 shows a chart demonstrating an area of the oxidized ApoA-I peptide (W (dioxidation: corresponding to the 72nd amino acid residue of SEQ ID NO: 1) in the amino acid sequence: EQLGPVTQEFWDNLEK (SEQ ID NO: 2; corresponding to the amino acid sequence consisting of the 62nd to 77th amino acids of SEQ ID NO: 1), hereafter, it may be referred to as the “dioxidized W72 peptide”) in 1 μm of a serum sample determined by analysis of oxidized proteins in HDLs in clinical samples (a group of healthy subjects (H), a group of SS patients (SS), and a group of NASH subjects (NASH)).

FIG. 2 shows a chart demonstrating an area of total ApoA-I in 1 μm of a serum sample determined by analysis of oxidized proteins in HDLs in clinical samples (a group of healthy subjects (H), a group of SS patients (SS), and a group of NASH subjects (NASH)).

FIG. 3 shows a chart demonstrating values determined by dividing the area of the dioxidized W72 peptide by the area of total ApoA-I in 1 μm of a serum sample determined by analysis of oxidized proteins in HDLs in clinical samples (a group of healthy subjects (H), a group of SS patients (SS), and a group of NASH subjects (NASH)).

FIG. 4 shows a chart demonstrating the results of AUC analysis of the dioxidized W72 peptide performed for evaluation of NASH diagnostic performance using the SS group and the NASH group.

FIG. 5 shows a chart demonstrating the results of AUC analysis of total ApoA-I performed for evaluation of NASH diagnostic performance using the SS group and the NASH group.

FIG. 6 shows a chart demonstrating the results of AUC analysis of the dioxidized W72 peptide/total ApoA-I ratio performed for evaluation of NASH diagnostic performance using the SS group and the NASH group.

FIG. 7 shows a chart demonstrating generation of TBARS in HDLs oxidized by copper sulfate at various concentrations: Condition 1: the final concentration of copper sulfate: 2.5 μM; Condition 2: the final concentration of copper sulfate: 0.5 μM; Condition 3: the final concentration of copper sulfate: 0.1 μM; Condition 4: the final concentration of copper sulfate: 0.02 μM.

FIG. 8 shows a chart demonstrating an area of the oxidized ApoA-I peptide (W (oxidation: corresponding to the 72nd amino acid residue of SEQ ID NO: 1) in the amino acid sequence: EQLGPVTQEFWDNLEK (SEQ ID NO: 2; corresponding to the amino acid sequence consisting of the 62nd to 77th amino acids of SEQ ID NO: 1), hereafter, it may be referred to as the “monooxidized W72 peptide”) in HDLs oxidized by copper sulfate at various concentrations.

FIG. 9 shows a chart demonstrating an area of dioxidized W72 peptide in HDLs oxidized by copper sulfate at various concentrations.

### EMBODIMENTS OF THE INVENTION

Hereafter, the present invention is described in detail.

The method of testing for nonalcoholic fatty liver disease (NAFLD) according to the present invention (hereafter, referred to as “the method of the present invention”) comprises a step of measuring the abundance of the oxidized apolipoprotein A-I (ApoA-I) in a biological sample obtained from a patient with NAFLD. The method of the present invention can also be referred to as, for example, a method for diagnosis of NAFLD, a method for assisting diagnosis of NAFLD, or a method for in vitro data collection for testing of NAFLD.

The present inventors found that the abundance of the oxidized ApoA-I, in particular, the oxidized ApoA-I modified by dioxidation of the 72nd tryptophan (tryptophan at position 72) in the ApoA-I consisting of the amino acid sequence as shown in SEQ ID NO: 1 (hereafter, referred to as “dioxidized W72 ApoA-I”), in a patient with nonalcoholic steatohepatitis (NASH) would be significantly higher than that in a patient with simple steatosis (SS). This has led to the completion of the present invention. The oxidized ApoA-I (dioxidized W72 ApoA-I, in particular) can be a diagnostic biomarker of NASH (or a diagnostic biomarker of NAFLD) used to differentiate NASH from SS.

In the dioxidized W72 ApoA-I, the 72nd tryptophan residue is dioxidized and converted into N-formylkynurenine or dihydroxytryptophan.

In the method of the present invention, any biological sample containing ApoA-I may be used, and examples include high-density lipoproteins (HDLs) in blood samples, such as whole blood, serum, and plasma samples, with a serum sample being preferable.

HDLs can be isolated from a serum sample by, for example, a method described below. Specifically, HDLs are collected from a serum sample (200 to 500 μl) via ultracentrifugation and gel filtration HPLC (Sakurai T. et al., Ann. Clin. Biochem., 49: 456-62, 2012). At the outset, the specific gravity of the serum “d” is adjusted to 1.225 g/ml with the use of potassium bromide, and a specific gravity liquid (d=1.225 g/ml) is added to bring the total amount of the solution to 8 ml. Samples are mounted on a rotor (MLN-80) and centrifuged using an ultracentrifuge (Optima MAX Ultracentrifuge, Beckman Coulter) (50,000 rpm, 20 hours, 4° C.). A supernatant (2.5 ml) containing a total lipoprotein fraction is collected, concentrated using a centrifuge tube provided with a ultrafiltration membrane filter of a molecular weight of 50 kDa (Amicon Ultra-0.5), and subjected to gel filtration HPLC using Superose 6 columns (Shimadzu Corporation) to isolate HDLs. HPLC is performed with the use of, as an eluate, 50 mM PBS (pH 7.4) containing 150 mM NaCl.

Subsequently, the isolated HDLs may be subjected to protein quantification. For example, the protein concentration in an HDL fraction may be quantified by the modified Lowry method (Markwell M. A. et al., Ann. Biochem., 87: 206-10, 1978). A solution (750 μl) comprising Solution A (2% Na₂CO₃, 0.4% NaOH, 0.16% sodium tartrate, 1% SDS) and Solution B (4% CuSO₄·5H₂O) mixed at 100:1 is added to 250 μl of a sample or standard solution (bovine serum albumin), and the resultant is incubated at room temperature for 30 minutes. After incubation, a solution comprising a phenol reagent and deionized water mixed at 1:1 is added in an amount of 75 μl each to the sample or standard solution with strong stirring using a vortex, and the resultant is incubated at room temperature for 45 minutes. After incubation, the absorbance (660 nm) is measured using a spectrophotometer (V-530, Jasco Corp.), the concentration and the absorbance of the standard solution are plotted to prepare a calibration curve, and the protein concentration of the sample can be determined based on the calibration curve.

In the method of the present invention, the abundance of the oxidized ApoA-I (dioxidized W72 ApoA-I, in particular) in a biological sample obtained from a patient with NAFLD, such as HDL in a blood sample, is to be measured.

For example, the abundance of the oxidized ApoA-I in the HDLs in the blood sample may be measured in the manner described below. Specifically, HDL is subjected to reductive alkylation of a protein by DTT and IAA, and the protein is treated with trypsin at 37° C. overnight for protein fragmentation. A peptide resulting from fragmentation is subjected to mass analysis using Orbitrap in accordance with a conventional technique (Sakurai T. et al., J. Sci. Food Agric., 99: 1675-81, 2019). As a result of this analysis, as the oxidized ApoA-I peptide, the peptide having the amino acid sequence: EQLGPVTQEFWDNLEK (SEQ ID NO: 2; corresponding to the amino acid sequence consisting of the 62nd to 77th amino acids of SEQ ID NO: 1) with dioxidized W (corresponding to the 72nd amino acid residue in SEQ ID NO: 1) in such an the amino acid sequence (“the dioxidized W72 peptide”) would be detected. The area deduced based on the spectral region of the dioxidized W72 peptide is determined by correcting a dilution factor in the process of analysis and in terms of a value in 1 μl of a blood sample, and the determined value of the dioxidized W72 peptide may be adopted as the abundance of the oxidized ApoA-I. Also, the determined value of the dioxidized W72 peptide may be divided by the value of the total ApoA-I in 1 μl of a blood sample (i.e., the abundance of the total ApoA-I) also obtained via mass analysis, and the calculated value may be represented as the ratio of the abundance of the oxidized ApoA-I relative to the abundance of the total ApoA-I (oxidized ApoA-I/total ApoA-I).

In the method of the present invention, the measured abundance of the oxidized ApoA-I is used as the indicator to identify whether NAFLD from which a patient suffers is NASH or SS.

When the abundance of the oxidized ApoA-I measured is significantly higher (for example, 1.74 times higher) than the abundance in the biological sample obtained from a patient known to have SS, for example, the NAFLD patient from which the biological sample has been obtained can be determined to have a higher possibility of having developed NASH. When the abundance of the oxidized ApoA-I measured is significantly lower than the abundance in the biological sample obtained from a patient known to have NASH, in other words, the NAFLD patient from which the biological sample has been obtained can be determined to have a higher possibility of having developed SS.

On the basis of the abundance of the oxidized ApoA-I measured, in addition, the ratio of the abundance of the oxidized ApoA-I as measured to the abundance of total ApoA-I in the biological sample (oxidized ApoA-I/total ApoA-I) may be used as the indicator to identify whether NAFLD from which a patient suffers is NASH or SS. When such ratio is significantly higher (e.g., 5.42 times higher) than the ratio in the biological sample obtained from a patient known to have SS, the patient of interest can be determined to have a higher possibility of having developed NASH. When such ratio is significantly lower than the ratio in the biological sample obtained from a patient known to have NASH, in other words, the patient of interest can be determined to have a higher possibility of having developed SS.

### EXAMPLES

Hereafter, the present invention is described in greater detail with reference to the Examples, although the technical scope of the present invention is not limited to these Examples.

### 1. Method

### 1-1. Collection of Samples

In order to perform profile analysis of oxidized proteins in the metal-oxidized HDLs, healthy subjects were subjected to blood sampling in the fasting state to obtain serum samples of healthy subjects. In order to analyze oxidized proteins in HDLs in actual clinical samples, in addition, serum samples of healthy subjects (n=6), SS patients (n=6), and NASH patients (n=10) provided by the Department for Gastroenterological/Liver Internal Medicine, Okayama University Hospital were used. SS patients and NASH patients were subjected to histopathological diagnosis by hepatobiopsy (NAFLD activity score (>5 points) and the Brunt scoring system). Healthy subjects were volunteer subjects with no history of liver diseases. The ethics of the research was approved at Okayama University (Approved Number: 1604-011), Okayama Municipal City Hospital (30-2), and Hokkaido University (17-33, 18-69-2).

### 1-2. Separation of Serum Lipoproteins

HDLs were collected from serum samples (200 to 500 μl) via ultracentrifugation and gel filtration HPLC (Sakurai T. et al., Ann. Clin. Biochem., 49: 456-62, 2012). At the outset, the specific gravity of the serum “d” was adjusted to 1.225 g/ml with the use of potassium bromide, and a specific gravity liquid (d=1.225 g/ml) was added to bring the total amount of the solution to 8 ml. Samples were mounted on a rotor (MLN-80) and centrifuged using an ultracentrifuge (Optima MAX Ultracentrifuge, Beckman Coulter) (50,000 rpm, 20 hours, 4° C.). A supernatant (2.5 ml) containing a total lipoprotein fraction was collected, concentrated using a centrifuge tube provided with an ultrafiltration membrane filter of a molecular weight of 50 kDa (Amicon Ultra-0.5), and subjected to gel filtration HPLC using Superose 6 columns (Shimadzu Corporation) to isolate HDLs. HPLC was performed with the use of, as an eluate, 50 mM PBS (pH 7.4) containing 150 mM NaCl. The HDLs obtained herein were designated as “unoxidized HDLs (native HDLs, nHDLs).”

### 1-3. Protein Quantification of HDL

The protein concentration in an HDL fraction was quantified by the modified Lowry method (Markwell M. A. et al., Ann. Biochem., 87: 206-10, 1978). A solution (750 μl) comprising Solution A (2% Na₂CO₃, 0.4% NaOH, 0.16% sodium tartrate, 1% SDS) and Solution B (4% CuSO₄·5H₂O) mixed at 100:1 was added to 250 μl of a sample or standard solution (bovine serum albumin), and the resultant was incubated at room temperature for 30 minutes. After incubation, a solution comprising a phenol reagent and deionized water mixed at 1:1 was added in an amount of 75 μl each to the sample or standard solution with strong stirring using a vortex, and the resultant was incubated at room temperature for 45 minutes. After incubation, the absorbance (660 nm) was measured using a spectrophotometer (V-530, Jasco Corp.), the concentration and the absorbance of the standard solution were plotted to prepare a calibration curve, and the protein concentration of the sample was determined based on the calibration curve.

### 1-4. Preparation of Oxidized HDL

With reference to the previous reports, oxidized HDLs were prepared (Hui S. P. et al., Anal. Bioanal. Chem., 403: 1831-40, 2012). Specifically, 4 μl of a CuSO₄ solution (final concentration: 0.02, 0.1, 0.5, or 2.5 μM) was added to 150 μl of an HDL solution with the protein concentration of 0.04 mg/ml. The resultant was incubated at 37° C. for 0 to 24 hours, and 5 μl each of 1 mM EDTA solution was added 0, 2, 8, and 24 hours later to terminate oxidation. Thus, an oxHDL solution was obtained. Thereafter, the oxidized states of nHDL and oxHDL at each oxidation time were evaluated by the thiobarbituric acid (TBA) method. Assay was performed using the TBARS assay kit (Cayman) (Watanabe M., J. Agric. Food Chem., 60: 830-5, 2012).

### 1-5. Proteomics

The amount of proteins in each HDL sample was adjusted to the same level of 5 μg via dilution. Proteins were subjected to reductive alkylation by DTT and IAA, and the protein was treated with trypsin at 37° C. overnight. The samples were solidified to dryness, dissolved again with 0.1% trifluoroacetic acid in water, and then desalted with Zip-Tip. The peptide derived from the sample was eluted with 0.1% formic acid in water, solidified to dryness, and then stored at −80° C. The peptide was dissolved again with 20 μl of 0.1% formic acid in water and then subjected to mass analysis using Orbitrap in accordance with a conventional technique (Sakurai T. et al., J. Sci. Food Agric., 99: 1675-81, 2019). The Thermo EASY-nLC Orbitrap Elite apparatus (Thermo Fisher Scientific) comprising the Fourier-transform and ion-trap mass spectrometers connected thereto was used. The peptides exhibiting the 10 strongest ion intensities detected per unit time were analyzed for 65 minutes. The Thermo Acclaim PepMap 100 (C18) separation columns and the NANO HPLC CAPILLARY COLUMN sprays (NTCC-360, NIKKYO TECHNOS CO., LTD) were used. A gradient solvent system was employed (Solvent A: 0.1% formic acid in water; Solvent B: 0.1% formic acid in CAN). The amount of the sample injected was 10 μl and sample injection was performed at a flow rate of 20 μl/min. Alignment was performed with the use of SEQUEST (version 1.4.1.14, Thermo Fisher Scientific) and the human amino acid sequence database (SwissProt). With the use of Proteome Discoverer software (version 1.3.0, Thermo Fisher Scientific), unoxidized and oxidation-modified peptides were analyzed. Specifically, analysis was performed to include, as variable modifications, oxidation (M, W, K), deamidation (NQ), carbamidomethylation (C), and acetylation (N terminus). The area deduced based on the spectral region of the target peptide in each sample was determined by correcting the dilution factor in the process of analysis and in terms of the value in 1 μl of the serum sample. Separately, the corrected peptide value was divided by the value of the total ApoA-I obtained via mass analysis in the same manner and calculated as the target peptide content as contained in Apo A-I.

### 1-6. Statistics

As statistical software, GraphPad Prism (San Diego, CA) was used. Comparison was performed among 3 groups by the Tukey's multiple comparison test. In order to investigate the capacity for differentiation of the NASH group from the SS group, the area under the curve (AUC) was determined based on the ROC curves each indicating relevant performance.

### 2. Results

### 2-1. Analysis of Oxidized Protein in HDL in Clinical Sample

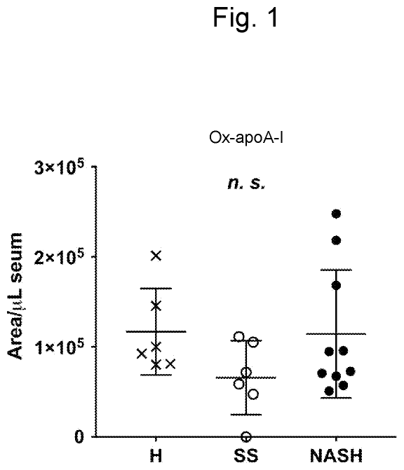

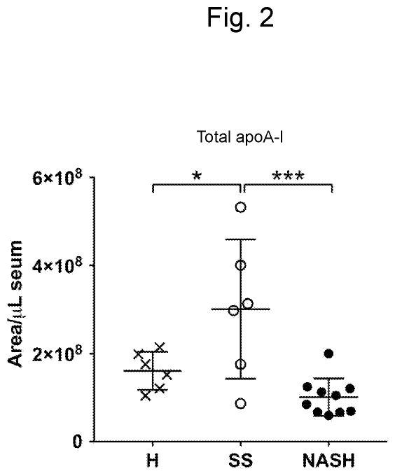

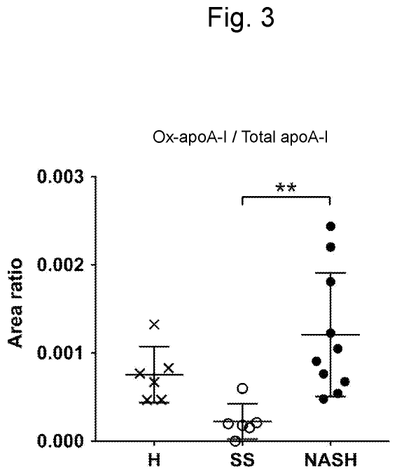

Concerning the area of the target peptide in 1 μl of the serum sample, the oxidized ApoA-I peptide that was detected at a significantly higher level in the NASH group compared with the SS group was limited to W (dioxidation: corresponding to the 72nd amino acid residue in SEQ ID NO: 1) in the amino acid sequence EQLGPVTQEFWDNLEK (SEQ ID NO: 2; corresponding to the amino acid sequence consisting of the 62nd to 77th amino acids in SEQ ID NO: 1) (the dioxidized W72 peptide). Such peptide was detected at levels 1.74 times (not significant) and 1.78 times (not significant) higher in the NASH group and in the healthy group than in the SS group, respectively (FIG. 1). Total ApoA-I was detected at levels 0.34 times (P<0.001) and 0.538 times (P<0.05) in the NASH group and in the healthy group than in the SS group, respectively (FIG. 2). Concerning the dioxidized W72 peptide, the values obtained by dividing the area of the target peptide by the total ApoA-I in 1 μl of the serum sample were 5.43 times (p<0.01) and 3.39 times (not significant) higher in the NASH group and in the healthy group than in the SS group, respectively (FIG. 3).

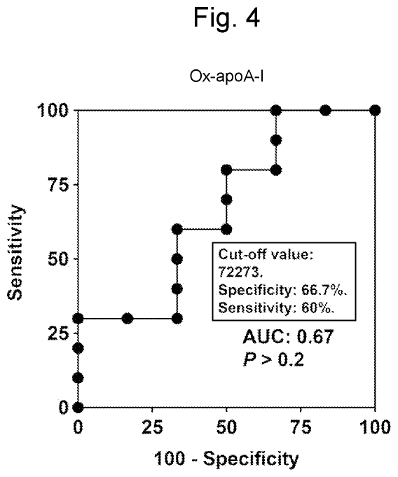

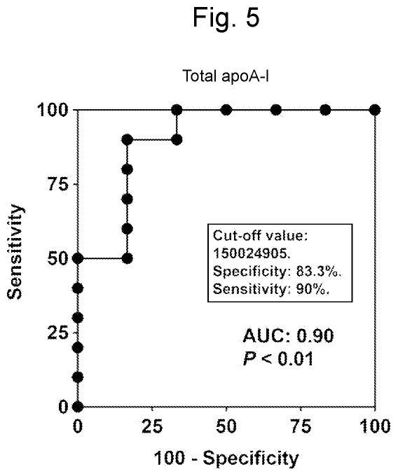

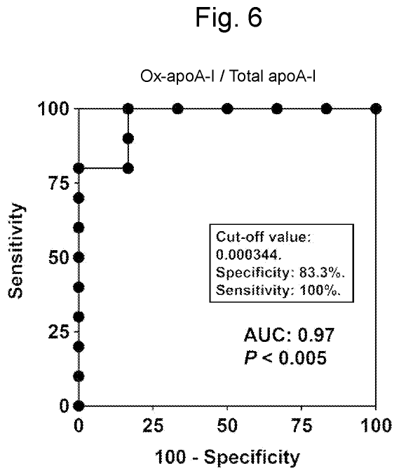

In AUC analysis aimed at evaluation of the capacity for diagnosis of NASH using the SS group and the NASH group, AUC in terms of the area of the target peptide in 1 μl of the serum sample was 0.67 (not significant), the cut-off value was 72273, the sensitivity was 60%, and the specificity was 66.7% (FIG. 4). AUC in terms of the peak area of the total ApoA-I was 0.87 (P<0.05), the cut-off value was 150024905, the sensitivity was 90%, and the specificity was 83.3% (FIG. 5). AUC in terms of the ratio of the oxidation-modified peptide/total ApoA-I was 0.97 (P<0.005), the cutoff value was 0.000344, the sensitivity was 100%, and the specificity was 83.3%. That is, the capacity for diagnosis was found to be good (FIG. 6).

### 2-2. Profile Analysis of Oxidized Proteins in Metal-Oxidized HDL

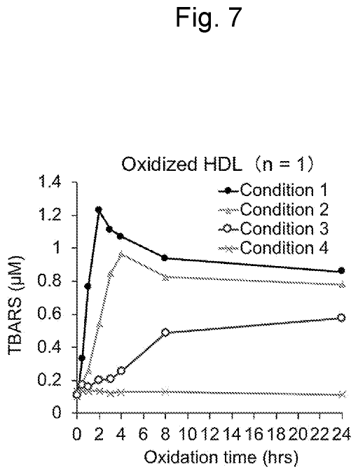

The above-mentioned dioxidized W72 peptide is considered to reflect the oxidized state in blood. Whether artificial oxidation of HDLs in healthy subjects would produce such particular oxidation-modified peptide was inspected herein. At the outset, an extent of oxidation of HDL by copper sulfate was first inspected using TBARS (FIG. 7). When the final concentration of copper sulfate was 0.02 μM, the TBARS value did not increase within 24 hours after the initiation of this oxidation. When the final concentration of copper sulfate was 0.1 μM, the TBARS value linearly increased as the oxidation time was prolonged. When the final concentration of copper sulfate was 2.5 μM, the TBARS concentration increased 12.0 times to reach the peak 2 hours after the initiation of oxidation compared to nHDL, and reached a plateau within 2 to 24 hours.

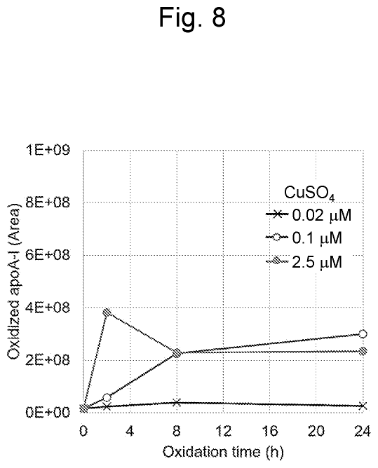

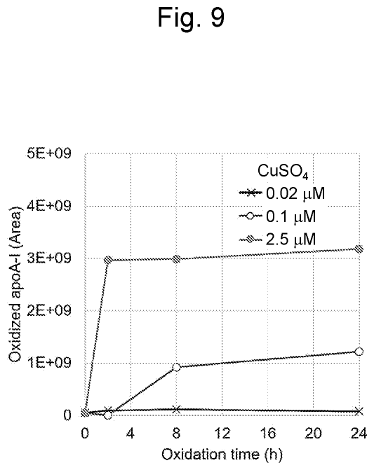

Concerning the samples under the same conditions, the area of W (oxidation) of EQLGPVTQEFWDNLEK (monooxidized W72 peptide) increased in a copper sulfate concentration-dependent and oxidation time-dependent manner. In the group with the highest copper sulfate concentration, however, the area reached the peak within 2 hours and was then lowered (FIG. 8). The area of the dioxidized W72 peptide increased in a copper sulfate concentration-dependent and oxidation time-dependent manner (FIG. 9).

### 2-3. Correlation Between Pathological Diagnosis of Liver Tissue and the Ratio of Oxidation-Modified Peptide (Dioxidized W72 Peptide)/Total ApoA-I

Pathological diagnosis of liver tissue obtained via hepatic biopsy was performed using the NAFLD activity scores (NAS). The correlation between each score of the pathological diagnosis and the value of the ratio of oxidation-modified peptide (dioxidized W72 peptide)/total ApoA-I was inspected. The results are shown in Table 1 below.

On the basis of the pathological scores, patients were divided into groups, and whether or not there would be differences in the value of the ratio of oxidation-modified peptide (dioxidized W72 peptide)/total ApoA-I between groups was inspected by the T test. At higher lobular inflammation, ballooning, and fibrosis scores, the ratio of oxidation-modified peptide (dioxidized W72 peptide)/total ApoA-I was found to be significantly higher. No significant differences were observed between groups in terms of the Steatosis scores.

TABLE 1
Associations of ox-apoAI/apoAI with histological findings

| | | Number of | Mean of | |
|---|---|---|---|---|
| Histological finding | Score | patients | ox-apoAI/apoAI | P value |
| Steatosis | 0/1 | 8 | 0.00083 | 0.9486 |
| | 2/3 | 8 | 0.00085 | |
| Lobular inflammation | 0 | 7 | 0.00036 | 0.0165 |
| | 1/2 | 9 | 0.00121 | |
| Ballooning | 0 | 8 | 0.00035 | 0.0035 |
| | 1/2 | 8 | 0.00133 | |
| Fibrosis | 0 | 7 | 0.00027 | 0.0026 |
| | 1/2/3/4 | 9 | 0.00128 | |

### 3. Discussion

In the experiments involving the use of clinical species, an increase of the dioxidized W72 peptide of ApoA-I in HDL was observed in the NASH group. This is deemed to mean that HDLs of the NASH group are more oxidized than those of other groups. In addition, the AUC value indicating the differentiation capacity was confirmed to be satisfactory. This indicates that this target peptide may be able to serve as a biomarker in blood that can differentiate NASH from SS.

In the experiment involving the use of artificial copper-oxidized HDLs, the dioxidized W72 peptide of ApoA-I in HDLs increased in added copper sulfate concentration-dependent and oxidation time-dependent manner. Once this target peptide was generated, the level thereof was not lowered. This suggests that such target peptide may serve as a stable marker and it was considered that it may serve as one of oxidized HDL indicators. Meanwhile, the level of W (oxidation) of this peptide was decreased as the extent of oxidation was increased. Such phenomena may have occurred because oxidation had further advanced to the state of dioxidation.

As shown in Table 1, the correlation was observed between the ratio of oxidation-modified peptide (dioxidized W72 peptide)/total ApoA-I and the pathological scores for lobular inflammation, ballooning, and fibrosis. Such pathological scores are used as signs that satisfactorily reflect the NASH liver. Therefore, this suggests that the use of the ratio of the method may be useful as the indicator so as to determine as to whether or not hepatic biopsy is to be performed.

The present research is expected to advance to the development of therapeutic agents targeting oxidized HDLs in the future, in addition to the development of the assay system targeting oxidized HDLs. It may be possible to prevent NASH by lowering the oxidized HDL level.

All publications, patents, and patent applications cited herein are incorporated herein by reference in their entirety.

1. A method of testing for nonalcoholic fatty liver disease comprising a step of measuring the abundance of the oxidized apolipoprotein A-I (ApoA-I) in a biological sample obtained from a patient with nonalcoholic fatty liver disease.

2. The method according to claim 1, wherein the step of measurement comprises measuring the abundance of the oxidized ApoA-I modified by dioxidation of the 72nd tryptophan of the ApoA-I consisting of the amino acid sequence as shown in SEQ ID NO: 1 in the oxidized ApoA-I in the biological sample obtained from the patient.

3. The method according to claim 1, which further comprises a step of testing for the nonalcoholic fatty liver disease of the patient to differentiate nonalcoholic steatohepatitis from simple steatosis using, as the indicator, the measured abundance of the oxidized ApoA-I.

4. The method according to claim 3, wherein the abundance greater than the abundance in the biological sample obtained from a patient with simple steatosis indicates a higher possibility of having developed nonalcoholic steatohepatitis.

5. The method according to claim 3, wherein the indicator is the ratio of the abundance of the oxidized ApoA-I relative to the abundance of total ApoA-I in the biological sample.

6. The method according to claim 5, wherein the ratio higher than the ratio in the biological sample obtained from a patient with simple steatosis indicates a higher possibility of having developed nonalcoholic steatohepatitis.

7. The method according to claim 1, wherein the biological sample obtained from the patient is a high-density lipoprotein (HDL) in a blood sample selected from the group consisting of whole blood, serum, and plasma samples.

8. A diagnostic biomarker of nonalcoholic steatohepatitis used to differentiate nonalcoholic steatohepatitis from simple steatosis comprising the oxidized apolipoprotein A-I (ApoA-I) modified by dioxidation of the 72nd tryptophan in the ApoA-I consisting of the amino acid sequence as shown in SEQ ID NO: 1.

9. The method according to claim 2, which further comprises a step of testing for the nonalcoholic fatty liver disease of the patient to differentiate nonalcoholic steatohepatitis from simple steatosis using, as the indicator, the measured abundance of the oxidized ApoA-I.

10. The method according to claim 9, wherein the abundance greater than the abundance in the biological sample obtained from a patient with simple steatosis indicates a higher possibility of having developed nonalcoholic steatohepatitis.

11. The method according to claim 9, wherein the indicator is the ratio of the abundance of the oxidized ApoA-I relative to the abundance of total ApoA-I in the biological sample.

12. The method according to claim 11, wherein the ratio higher than the ratio in the biological sample obtained from a patient with simple steatosis indicates a higher possibility of having developed nonalcoholic steatohepatitis.

13. The method according to claim 2, wherein the biological sample obtained from the patient is a high-density lipoprotein (HDL) in a blood sample selected from the group consisting of whole blood, serum, and plasma samples.


SEQUENCE LISTING (2 sequences)

SEQ ID NO: 1 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 16 aa, Homo sapiens
EQLGPVTQEFWDNLEK